(19) United States
(12) Patent Application Publication
(10) Pub. No.: US 2012/0051501 A1
(43) Pub. Date: Mar. 1, 2012

(54) RADIATION IMAGE RADIOGRAPHING APPARATUS AND RADIATION IMAGE RADIOGRAPHING AND DISPLAYING METHOD

(75) Inventor: Hiroki NAKAYAMA, Ashigarakami-gun (JP)

(73) Assignee: FUJIFILM Corporation, Tokyo (JP)

(21) Appl. No.: 13/215,485

(22) Filed: Aug. 23, 2011

(30) Foreign Application Priority Data
Aug. 24, 2010 (JP) 2010-186878

(51) Int. Cl. A61B 6/04; G01N 23/04; A61B 6/02

(52) U.S. Cl. 378/37; 378/41; 378/62

(57) ABSTRACT

It is possible to display a stereoscopic image in which a sense of depth in a stereoscopic image and a projection amount from a display screen are made uniform. A radiation image radiographing and displaying method irradiates radiation onto a subject from different radiographing directions by irradiation units to acquire radiation images in the radiographing directions detected by a radiation image detection unit, and displays a stereoscopic image using the acquired radiation images in the radiographing directions. The position of a subject is controlled such that the distance between the position of the end surface of the subject facing the irradiation units and the radiation image detection unit becomes a given distance, in a state where the position of the subject is controlled, the radiation images in the radiographing directions are acquired, and a stereoscopic image is displayed using the acquired radiation images.

### BACKGROUND OF THE INVENTION

1. Field of the Invention

The present invention relates to a radiation image radiographing apparatus and a radiation image radiographing and displaying method which display a stereoscopic image using radiation images detected by a radiation image detector through irradiation of radiation onto a subject from different directions.

2. Description of the Related Art

In the related art, a system is known in which a plurality of images are displayed in combination, and stereoscopic view can be realized using parallax. An image (hereinafter, referred to as a stereoscopic image or a stereo image) which can be viewed stereoscopically is generated based on a plurality of images having parallax obtained by radiographing the same subject from different positions.

Moreover, such way of generating stereoscopic image is utilized not only in the field of digital cameras and televisions but also in the field of capturing a stereoscopic radiological image. That is, a subject is irradiated with radiation from different directions, the radiation passing through the subject is detected by a radiological image detector to acquire plural radiological images having parallax, and a stereoscopic image is generated based on the radiological images. By generating a stereoscopic image in this way, a radiological image with a sense of depth can be observed and thereby more suitable radiological image for diagnosis can be observed.

However, when stereoscopic images of subjects are generated using radiation images obtained by radiographing subjects P1 and P2 having different thicknesses, as in a radiographing device in the related art shown in FIG. 9, if the distance between a right-eye radiation source 10R and a left-eye radiation source 10L irradiating radiation, that is, a base-line length L is set to the same value, and a subject is radiographed under the same image radiographing condition, since the thicknesses t1 and t2 of the subjects P1 and P2 are different from each other, the projection amount from a display screen of a stereoscopic image differs to be projected or retracted. For this reason, when an observer screens a stereoscopic image of a plurality of subjects P, there is a problem in that the eyes of the observer are tired. This problem also occurs when a stereoscopic image of portions having different thicknesses is generated.

Accordingly, when radiographing the subjects P1 and P2 having different thicknesses, as shown in FIG. 10, the value of the base-line length L for each subject varies to change the value of a convergence angle θ, or as shown in FIGS. 11A to 11C, when generating a stereoscopic image, the shift amounts of left and right images A1 and A2 change (JP1987-230194A (JP-S62-230194A)), such that the projection amount in a stereoscopic image becomes equal.

JP2005-168601A suggests that a distance obtained by adding the distance between two radiation sources to the upper part of a subject and half of the thickness of the subject is acquired, the interval (base-line length) between the two radiation sources is calculated based on the distance such that a desired convergence angle is obtained, and the two radiation sources are moved such that the interval is obtained.

As in the related art, however, in the method in which the convergence angle or the shift amounts of the left and right radiation images change to make the projection amount uniform, the convergence angle or the shift amounts of the left and right radiation images change to change parallax, and the ratio of the apparent absolute value when viewed from the depth direction (hereinafter, referred to as a sense of depth) changes. That is, when observing a stereoscopic image generated by radiographing a thin portion and a thick portion, if the projection amount is made uniform by the above-described method, it looks like the thickness is apparently the same, but actually, there is a phenomenon that the thin portion has a thickness of 2 mm and the thick portion has a thickness of 5 mm. With this phenomenon, there is a possibility that diagnosis is not easily performed, for example, at the time of medical diagnosis using a stereoscopic image.

### SUMMARY OF THE INVENTION

The present invention has been made in view of the above-mentioned problems and an object of the invention is to provide a radiation image radiographing apparatus and a radiation image radiographing and displaying method capable of displaying a stereoscopic image in which a sense of depth in a stereoscopic image and a projection amount from a display screen are made uniform.

An aspect of the invention provides a radiation image radiographing apparatus. The apparatus includes an irradiation unit which irradiates radiation onto a subject from different radiographing directions, a radiation image detection unit which detects radiation having been irradiated by the irradiation unit and having passed through the subject, and a subject positioning unit which controls the position of the subject such that the distance between the position of the end surface of the subject facing the irradiation unit and the radiation image detection unit becomes a given distance. The end surface refers to a surface which is perpendicular to a normal line from the detection surface of the radiation image detection unit toward the irradiation unit, and includes the position of the subject closest to the irradiation unit.

In the aspect of the present invention, the given distance refers to a value which is determined in advance before radiographing, and the effects of the present invention can be obtained in a stereoscopic image which is generated using the radiation images radiographed under the condition that, when observing stereoscopic images of subjects obtained by radiographing different subjects, the distance between the position of the end surface of the subject facing the irradiation unit and the radiation image detection unit is set to a given distance.

The radiation image radiographing apparatus may further include a subject position detection unit which detects the position of the end surface of the subject facing the irradiation unit. Moreover, the radiation image radiographing apparatus may further include a display device which acquires the radiation images in the radiographing directions detected by the radiation image detection unit and displays a stereoscopic image using the acquired radiation images in the radiographing directions;

The subject position detection unit may have an optical sensor, and the position of the end surface may be detected by the optical sensor.

The radiation image radiographing apparatus may further include a support portion which supports the subject, and a support portion driving unit which moves the position of the support portion between the irradiation unit and the radiation image detection unit. The subject positioning unit may control the support portion driving unit such that the distance between the position of the end surface of the subject and the radiation image detection unit becomes the given distance.

Another aspect of the present invention provides a breast image radiographing apparatus. The apparatus includes an irradiation unit which irradiates radiation onto the breast of a human subject from different radiographing directions, a radiation image detection unit which detects radiation having been irradiated by the irradiation unit and having passed through the breast, a support on which the breast is placed, a compression plate which is provided between the irradiation unit and the radiation image detection unit, and fixed at a given distance from the radiation image detection unit to compress the breast on the support, and a support driving unit which moves the position of the support toward the compression plate to compress the breast. Moreover, the breast image radiographing apparatus may further include a display device which acquires the radiation images in the radiographing directions detected by the radiation image detection unit and displays a stereoscopic image using the acquired radiation images in the radiographing directions;

A further aspect of the present invention provides a radiation image radiographing and displaying method which irradiates radiation onto a subject from different radiographing directions by an irradiation unit to acquire radiation images in the radiographing directions detected by the radiation image detection unit, and displays a stereoscopic image using the acquired radiation images in the radiographing directions. The position of the subject is controlled such that the distance between the position of the end surface of the subject facing the irradiation unit and the radiation image detection unit becomes a given distance, in a state where the position of the subject is controlled, the radiation images in the radiographing directions are acquired, and the stereoscopic image is displayed using the acquired radiation images.

A still further aspect of the present invention provides a breast image radiographing and displaying method which irradiates radiation onto the breast of a human subject from different radiographing directions by an irradiation unit to acquire radiation images in the radiographing direction detected by a radiation image detection unit, and displays a stereoscopic image using the acquired radiation images in the radiographing directions. A support is provided on which the breast is placed, and a compression plate is provided between the irradiation unit and the radiation image detection unit, and fixed at a given distance from the radiation image detection unit to compress the breast on the support. The position of the support is moved toward the compression plate to compress the breast, in a state where the breast is compressed, the radiation images in the radiographing directions are acquired, and the stereoscopic image is displayed using the acquired radiation images.

According to the radiation image radiographing apparatus and the radiation image radiographing and displaying method of the present invention, the position of the subject is controlled such that the distance between the position of the end surface of the subject facing the irradiation unit and the radiation image detection unit becomes a given distance, in a state where the position of the subject is controlled, the radiation images in the radiographing directions are acquired, and the stereoscopic image is displayed using the acquired radiation image. For this reason, when radiographing a plurality of subjects having difference thicknesses, the distance between the position of the end surface of the subject facing the irradiation unit and the radiation image detection unit becomes a given distance, regardless of which subject is radiographed, and even when the subjects are different in thickness, in the stereoscopic image of any subject, the projection amount on the stereoscopic image can be made uniform. Simultaneously, since the convergence angle does not change, or the shift amounts of the left-eye radiation image and the right-eye radiation image do not change at the time of the display of the stereoscopic image, there is no change in the parallax of the left-eye radiation image and the right-eye radiation image. For this reason, in the stereoscopic image of any subject, the sense of depth in the stereoscopic image can be made uniform.

The sense of depth, that is, the ratio of the absolute values of the eyes when viewed from the depth direction is not changed, such that, when observing a stereoscopic image generated by radiographing a thin portion and a thick portion, the apparent ratio of the thickness between the stereoscopic images becomes equal to the actual ratio, making it possible to facilitate diagnosis at the time of medical diagnosis.

According to the breast image radiographing apparatus and the breast image radiographing and displaying method of the present invention, the apparatus includes the support on which the breast is placed, and the compression plate which is provided between the irradiation unit and the radiation image detection unit, and fixed at a given distance from the radiation image detection unit to compress the breast on the support. The position of the support is moved toward the compression plate to compress the breast, in a state where the breast is compressed, the radiation images in the radiographing directions are acquired, and the stereoscopic image is displayed using the acquired radiation images. For this reason, when radiographing the breasts of human subjects having different thicknesses, the distance between the position of the compression plate and the radiation image detection unit becomes a given distance regardless of the breast of which human subject is radiographed, and even when the breast differs in thickness, in the stereoscopic image of any breast, the projection amount can be made uniform. Simultaneously, since the convergence angle does not change or the shift amounts of the left-eye radiation image and the right-eye radiation image do not change at the time of the display of the stereoscopic image, there is no change in the parallax of the left-eye radiation image and the right-eye radiation image. For this reason, in the stereoscopic image of any breast, the sense of depth in the stereoscopic image can be made uniform.

The sense of depth, that is, the ratio of the apparent absolute values in the depth direction is not changed, such that, when observing the stereoscopic image of a breast generated by radiographing a thin portion and a thick portion, the apparent ratio of the thickness between the stereoscopic images becomes equal to the actual ratio, making it possible to facilitate diagnosis at the time of medical diagnosis.

### BRIEF DESCRIPTION OF THE DRAWINGS

FIG. 1 is a schematic configuration diagram of a radiation stereo image radiographing apparatus using an embodiment of a radiation image radiographing apparatus of the present invention.

FIG. 2 is a block diagram showing the internal configuration of a radiation image detection unit and a computer in a radiation stereo image radiographing apparatus using an embodiment of a radiation image radiographing apparatus of the present invention.

FIG. 3 is a flowchart illustrating the action of a radiation stereo image radiographing apparatus using an embodiment of a radiation image radiographing apparatus of the present invention.

FIG. 4 is an explanatory view illustrating the action of a radiation stereo image radiographing apparatus using an embodiment of a radiation image radiographing apparatus of the present invention.

FIG. 5 is a schematic configuration diagram of a breast image radiographing apparatus using an embodiment of a breast image radiographing apparatus of the present invention.

FIG. 6 is a diagram of an arm portion in a breast image radiographing apparatus shown in FIG. 5 when viewed from the right direction of FIG. 5.

FIG. 7 is a block diagram showing the schematic internal configuration of a computer in a breast image radiographing apparatus shown in FIG. 5.

FIG. 8 is a flowchart illustrating the action of a breast image radiographing apparatus using an embodiment of a breast image radiographing apparatus of the present invention.

FIG. 9 is an explanatory view (first view) illustrating the action of a radiation stereo image radiographing apparatus in the related art.

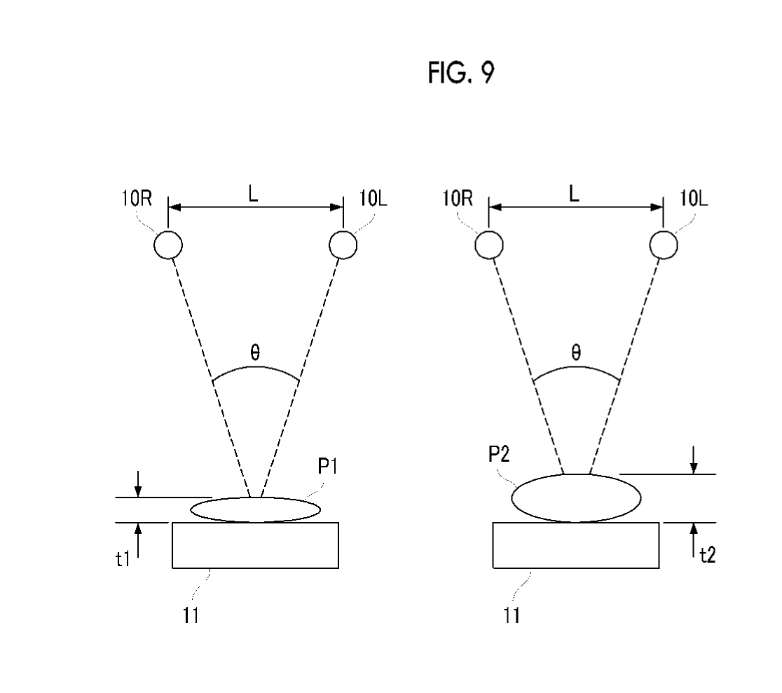

FIG. 10 is an explanatory view (second view) illustrating the action of a radiation stereo image radiographing apparatus in the related art.

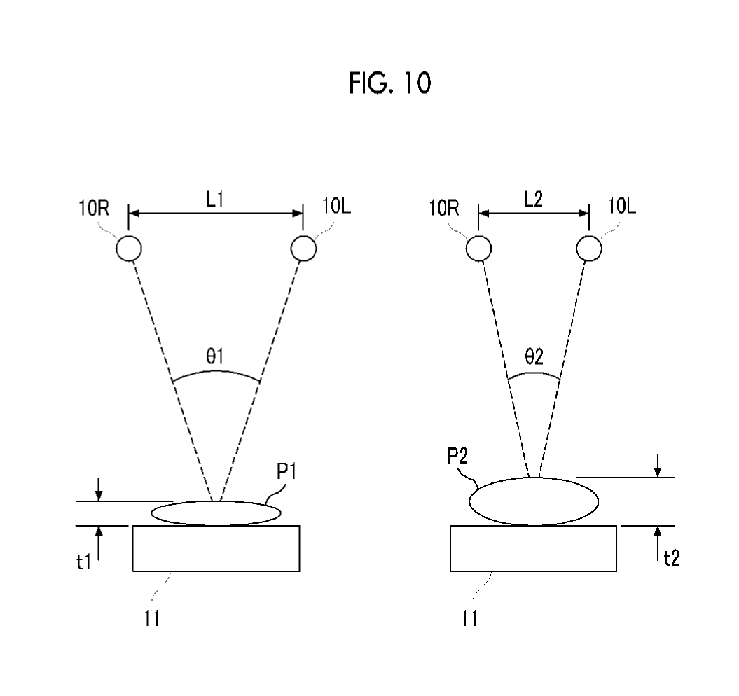

FIGS. 11A to 11C are explanatory views (third view) illustrating the action of a radiation stereo image radiographing apparatus in the related art.

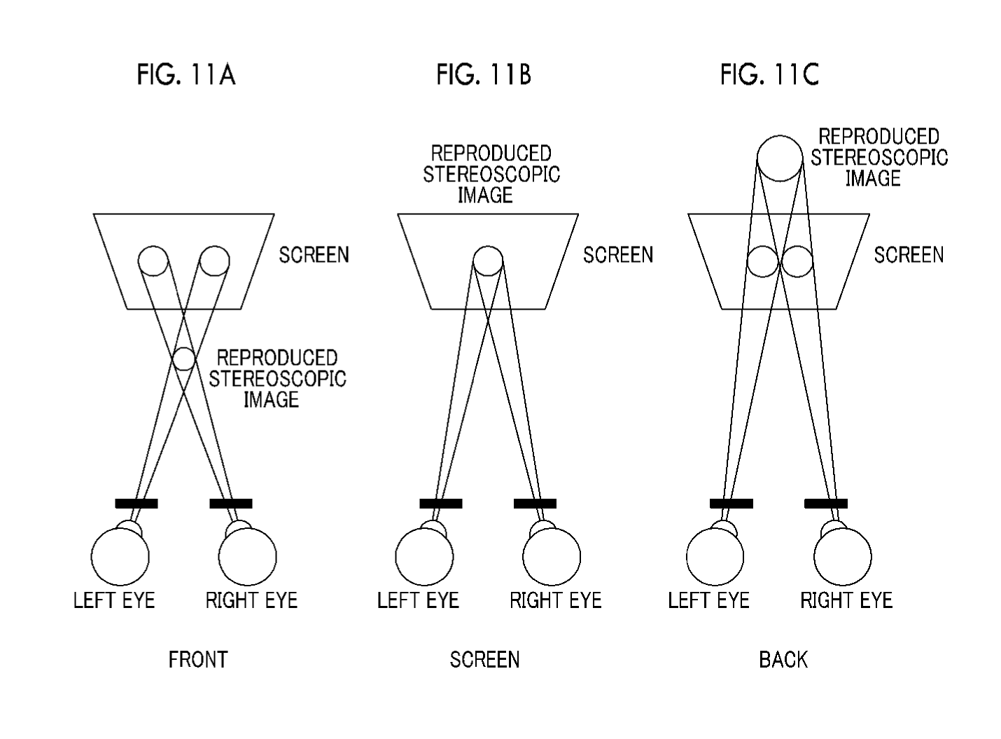

### DESCRIPTION OF THE PREFERRED EMBODIMENTS

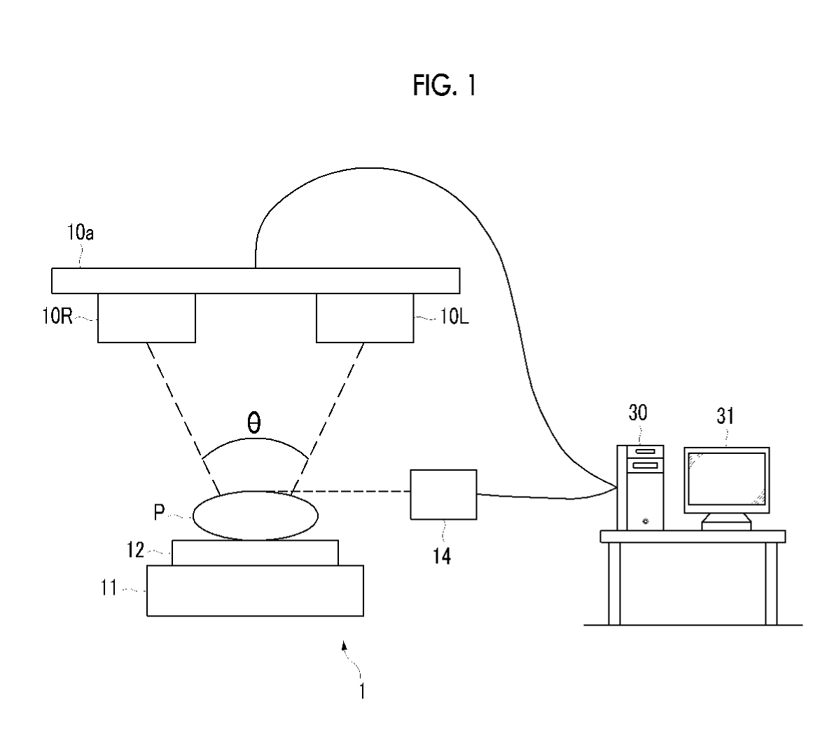

Hereinafter, a radiation stereo image radiographing apparatus using an embodiment of a radiation image radiographing apparatus of the present invention will be described with reference to the drawings. First, the schematic configuration of the entire radiation stereo image radiographing apparatus will be described. FIG. 1 is a diagram showing the schematic configuration of the radiation stereo image radiographing apparatus.

As shown in FIG. 1, the radiation stereo image radiographing apparatus includes a radiographing device 1 which radiographs the radiation images of a subject P, a computer 30 which is connected to the radiographing device 1, controls the radiographing device 1, and processes radiation image signal obtained by radiographing, and a monitor 31 which is connected to the computer 30.

The radiographing device 1 includes a left-eye radiation source 10L and a right-eye radiation source 10R which emit radiation, a radiation driving unit 10a which generates radiation from the left and right radiation sources 10L and 10R, a radiation image detection unit 11 which detects radiation emitted from the radiation sources 10L and 10R, a support 12 on which the subject P is placed, and an optical sensor 14 which detects the position of the uppermost surface of the subject P.

The left-eye radiation source 10L and the right-eye radiation source 10R are movably arranged on the radiation driving unit 10a, and the distance therebetween, that is, a base-line length is variable. If the base-line length varies, the convergence angle θ also varies with the variation in the base-line length.

The radiation driving unit 10a drives the left-eye and right-eye radiation sources 10L and 10R to generate radiation under a predetermined X-ray condition in accordance with an instruction from a radiographing control unit 30d described below.

Although in this embodiment, a case has been described where the two radiation sources 10L and 10R are used for radiographing, the present invention is not limited thereto, and a single radiation source 10 may be configured such that the emission direction of radiation varies for each radiographing.

The support 12 is provided on the upper surface of the radiation image detection unit 11 and moves in the up-down direction while the parallel state to the radiation image detection unit 11 is maintained by a support driving unit 12a described below. The movement in the up-down direction is carried out using a moving mechanism (not shown) which is generally used. Any mechanism may be used insofar as the mechanism can move in the up-down direction. The up direction refers to the radiation source side, and the down direction refers to the radiation image detection unit side.

The optical sensor 14 is provided near the support 12 or the radiation image detection unit 11, is placed on an installment base, and irradiates light in a direction parallel to the upper surface of the support 12 at a predefined position described below. When the support 12 on which the subject P is placed moves from below to above, and light is blocked by the subject P, the position of the uppermost surface of the subject P is detected.

Although in this embodiment, the optical sensor 14 is used to detect the position of the uppermost surface of the subject P, the present invention is not limited thereto, and for example, a contact sensor or the like may be used. Any means may be used insofar as the means can detect the position of the uppermost surface of the subject P.

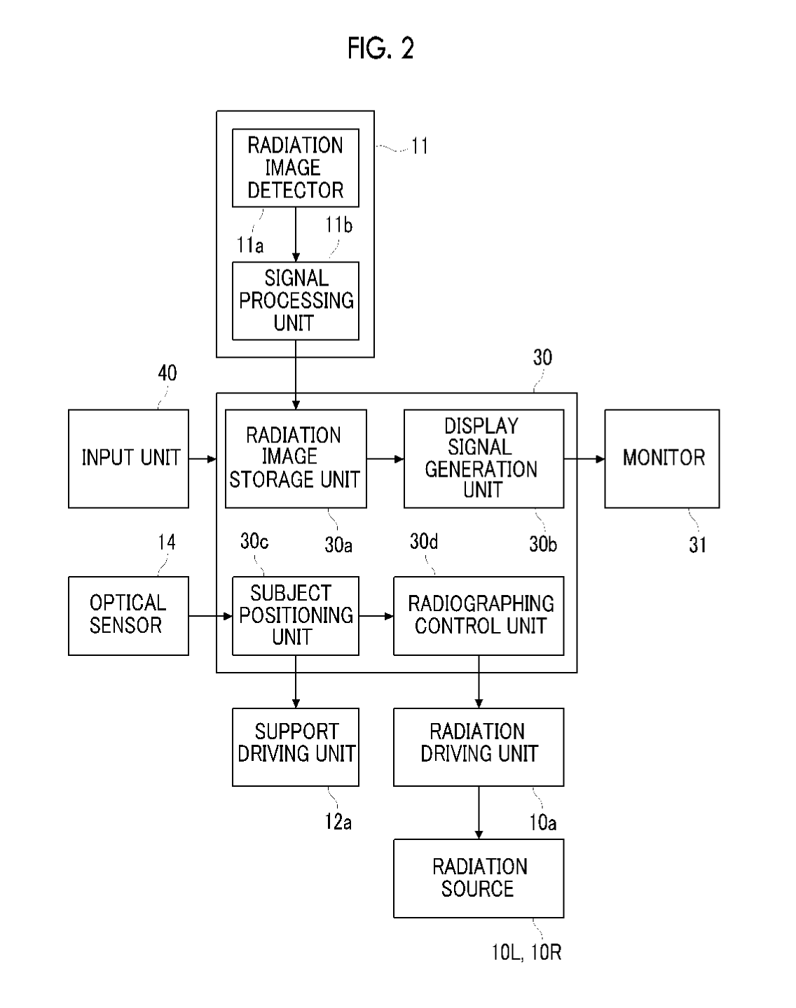

FIG. 2 is a block diagram showing the schematic internal configuration of the radiation image detection unit 11 and the computer 30.

As shown in FIG. 2, the radiation image detection unit 11 includes a radiation image detector 11a which receives radiation having passed through the subject P to generate electric charges, and outputs radiation image signals representing the radiation images of the subject P, and a signal processing unit 11b which performs predetermined signal processing on the radiation image signals output from the radiation image detector 11a.

The radiation image detector 11a can repeatedly record and read the radiation images, and may be a so-called direct-type radiation image detector which directly receives radiation to generate electric charges, or a so-called indirect-type radiation image detector which temporarily converts radiation to visible light and converts visible light to an electric charge signal. As a method of reading radiation image signals, it is preferable to use a so-called TFT reading method which turns on/off a TFT (thin film transistor) switch to read radiation image signals or a so-called light reading method which irradiates read light to read radiation image signals. The present invention is not limited thereto, and other methods may be used.

The signal processing unit 11b includes an amplifier unit which has a charge amplifier for converting an electric charge signal read from the radiation image detector 11a to a voltage signal, an AD conversion unit which converts the voltage signal output from the amplifier unit to a digital signal, and the like.

The computer 30 includes a central processing unit (CPU), a storage device, such as a semiconductor memory, a hard disk, or an SSD, and the like, which constitute a radiation image storage unit 30a, a display signal generation unit 30b, a subject positioning unit 30c, and a radiographing control unit 30d.

The radiation image storage unit 30a stores radiation image signals at radiographing angles detected by the radiation image detection unit 11.

The display signal generation unit 30b generates a display control signal based on two radiation image signals read from the radiation image storage unit 30a and outputs the display control signal to the monitor 31.

The subject positioning unit 30c instructs the support driving unit 12a to drive the support 12 such that the support 12 on which the subject P is placed moves upward, and if a detection signal regarding the position of the uppermost surface of the subject P is input from the optical sensor 14, instructs the support driving unit 12a to stop the support such that the movement of the support stops. The position where the optical sensor 14 irradiates light is set to a position distant from the upper surface of the support 12 when the support 12 is at the lowermost position by a value greater than the normal thickness of the subject P.

The radiographing control unit 30d controls the irradiation timing of radiation emitted from the radiation sources 10L and 10R, and controls the radiation generation conditions (tube current, time, tube current-time product, and the like) in the radiation sources 10L and 10R.

An input unit 40 has, for example, a keyboard or a pointing device, such as a mouse, and receives an input of a radiographing condition of a radiographer or an input of a radiographing start instruction.

The monitor 31 displays a stereo image using two radiation image signals output from the computer 30, and can have a configuration in which the radiation images based on the two radiation image signals are respectively displayed on two screens, and a half mirror, a polarizing glass, or the like is used such that one radiation image is incident on the right eye of the observer and another radiation image is incident on the left eye of the observer, thereby displaying a stereo image. A configuration may be made in which two radiation images are superimposingly displayed while shifting by a predetermined parallax amount and observed by a polarizing glass, thereby generating a stereo image. A configuration may be made in which, like a parallax barrier method and a lenticular method, two radiation images are displayed on a 3D liquid crystal device which can be viewed stereoscopically, thereby generating a stereo image.

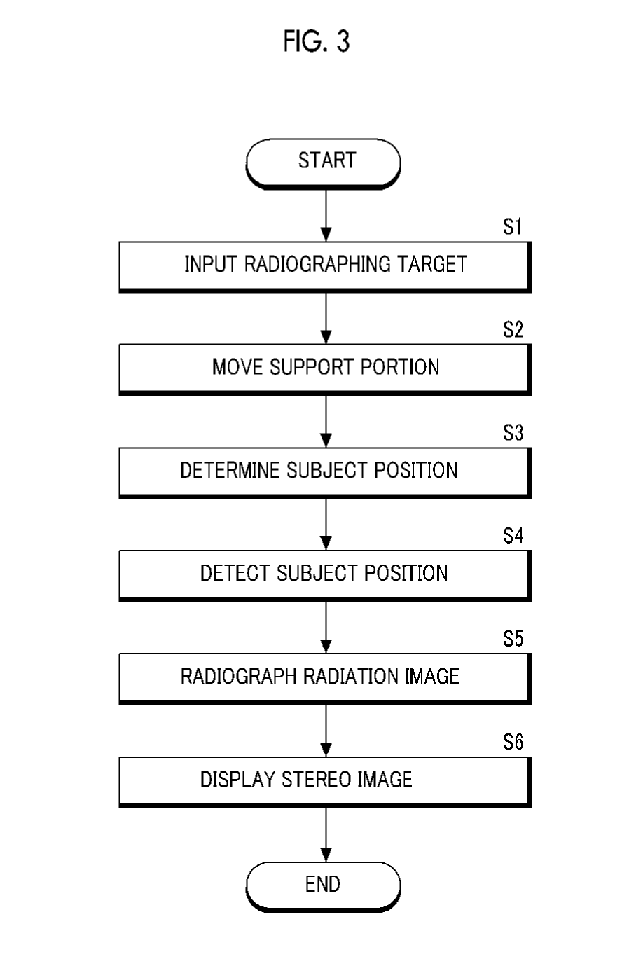

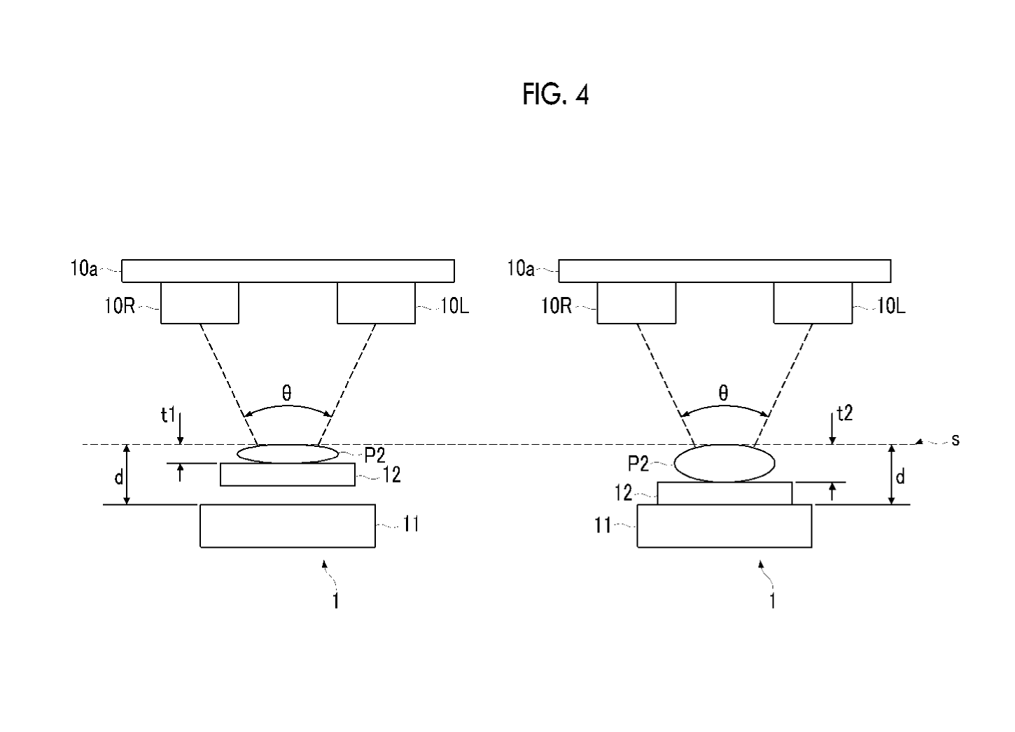

Next, the action of the radiation stereo image radiographing apparatus will be described with reference to a flowchart of FIG. 3 and FIG. 4.

First, as shown in FIG. 1, the subject P is placed on the support 12. A radiographer inputs information regarding a radiographing target, such as a head, a cervical spine, stomach, lungs, an abdomen, or a breast, using the input unit 40 (S1).

In the computer 30, a radiographing condition table in which various radiographing targets are associated with radiographing conditions is set in advance. In the radiographing condition table, as the radiographing conditions, for example, the targets of the radiation sources 10L and 10R, the types of filters, the tube voltages of the radiation sources 10L and 10R, mAs value, and the like are set. The value of a convergence angle θ for radiographing a stereo image is also set. In the embodiment of the present invention, the value of the convergence angle θ has a given value defined in advance for each radiographing target.

If a radiographing target is input from the input unit 40, the above-described radiographing condition table is referenced, the conditions of the radiation sources 10L and 10R, and the like are acquired. Simultaneously, information regarding the convergence angle θ is acquired. The conditions are input to the radiographing control unit 30d. In this embodiment, it is assumed that θ=±2° is set in advance as information regarding the convergence angle θ, and the radiation sources 10L and 10R are arranged based on the value of the convergence angle θ. In this embodiment, it is also assumed that two stereo images from two directions perpendicular to each other are radiographed and displayed.

It is assumed that the radiation image detection unit 11 is fixed at a position distant from the left-eye radiation source 10L and the right-eye radiation source 10R by a given distance defined in advance for each radiographing target.

If a radiographing start instruction is input by an operator, the optical sensor 14 is in an operation state, and the support 12 is moved in the up direction by the support driving unit 12a (S2). The optical sensor 14 detects the uppermost surface of the subject P and outputs a detection signal to the subject positioning unit 30c (S3).

The subject positioning unit 30c controls the support driving unit 12a to stop the movement of the support 12 based on the detection signal input from the optical sensor 14, and determines the radiographing position of the subject P (S4). That is, as shown in the left view of FIG. 4, in the case of a subject P1 having a small thickness, the support 12 moves in the up direction, and the support 12 stops at a position distant from the radiation image detection unit 11 compared to a subject P2 having a large thickness shown in the right view of FIG. 4. Although in the right view of FIG. 4, for convenience of description, in the case of the subject P2 having a large thickness, the support 12 and the radiation image detection unit 11 are in contact with each other, actually, the position S (a dotted line in the drawing) where the optical sensor 14 irradiates light is set to a position distant from the support 12 in a state where the support 12 and the radiation image detection unit 11 are in contact with each other by equal to or greater than the normal thickness of the subject, such that the support 12 and the radiation image detection unit 11 are not in contact with each other.

As described above, even when the subject P differs in thickness, the support 12 moves in the up direction by an amount corresponding to the thickness of the subject P, such that the distance d between the radiation image detection unit 11 and the uppermost surface of the subject P becomes a predetermined distance set in advance, that is, a given distance d regardless of the thickness of the subject P. Thus, even when the subject P differs in thickness, in any stereoscopic image, the projection amount on the stereoscopic image can be made uniform. Simultaneously, since the convergence angle θ does not change or the shift amounts of the left-eye radiation image and the right-eye radiation image do not change at the time of the display of the stereoscopic image, there is no change in the parallax of the left-eye radiation image and the right-eye radiation image. Thus, in the stereoscopic image of any subject P, the sense of depth in the stereoscopic image can be made uniform.

Although in this embodiment, the optical sensor 14 is used to detect the uppermost surface of the subject P, the present invention is not limited thereto. While the optical sensor 14 is not used, thickness information of the subject P is acquired in advance and stored in the computer 30 by the input unit 40, and the subject positioning unit 30c reads the thickness information, calculates the movement distance of the support 12 based on the thickness information, and outputs the movement distance to the support driving unit 12a. In this way, the distance between the uppermost surface of the subject P and the radiation image detection unit 11 can become the given distance d.

The radiographing position determined by the subject positioning unit 30c is output to the radiographing control unit 30d, and the radiographing control unit 30d outputs a control signal to the radiation driving unit 10a based on the input radiographing position to radiograph a radiation image for a right-eye stereo image and a radiation image for a left-eye stereo image (S5).

Specifically, when radiographing the radiation images for left-eye and right-eye stereo images, as shown in FIG. 1, radiation is sequentially emitted from the radiation sources 10L and 10R in which the radiographing angle θ=±2° is set.

The irradiation of conical radiation having passed through the subject P onto the radiation image detector 11a and the reading of the radiation image signals detected by the radiation image detector 11a are sequentially performed.

The read radiation image signals are converted to voltage signals by the amplifier unit of the signal processing unit 11b, converted to digital signals by the AD conversion unit, and input to and sequentially stored in the radiation image storage unit 30a.

Two radiation image signals for left-eye and right-eye stereo images are input to the display signal generation unit 30b, and the display signal generation unit 30b generates a display control signal based on the input radiation image signals and outputs the display control signal to the monitor 31. The monitor 31 displays the radiation images based on the input display control signal to display the left-eye stereo image and the right-eye stereo image (S6). The left-eye stereo image and the right-eye stereo image may be displayed simultaneously or switchingly.

When the observer observes the monitor 31, when the projection amount of the stereoscopic image is excessively projected or retracted and optimum stereoscopic view may not be performed, the shift amounts of the left-eye and right-eye stereo images may be shifted to adjust the projection amount. At this time, in this embodiment, with regard to a plurality of stereoscopic images obtained by radiographing a plurality of subjects P having different thicknesses, the projection amount in each stereoscopic image is made uniform, such that the shift amount at the time of the adjustment has the same value between the stereoscopic images, thereby maintaining the projection amount of each stereoscopic image uniform. Simultaneously, a change in the parallax amount between the left-eye and right-eye stereo image for generating each stereoscopic image can be made uniform in each stereoscopic image.

Therefore, in the stereoscopic images of a plurality of subjects P having different thicknesses, it is possible to adjust the projection amount while maintaining the projection amount and the sense of depth between the stereoscopic images uniform.

As described above, according to the radiation stereo image radiographing apparatus and the radiation stereo image radiographing and displaying method of this embodiment, the position of the subject P is controlled such that the distance between the position of the uppermost surface of the subject P and the radiation image detection unit 11 becomes a given distance defined in advance, in a state where the position of the subject P is controlled, the radiation images in the radiographing directions are acquired, and the stereoscopic image is displayed using the acquired radiation images. For this reason, when radiographing a plurality of subjects P having different thicknesses, the distance between the position of the uppermost surface of the subject P and the radiation image detection unit 11 becomes a given distance regardless of which subject P is radiographed, and even when the subject P differs in thickness, in the stereoscopic image of any subject P, the projection amount on the stereoscopic image can be made uniform. Simultaneously, since the convergence angle θ does not change or the shift amounts of the left-eye radiation image and the right-eye radiation image do not change at the time of the display of the stereoscopic image, there is no change in the parallax of the left-eye radiation image and the right-eye radiation image. For this reason, in the stereoscopic image of any subject, the sense of depth in the stereoscopic image can be made uniform.

The sense of depth, that is, the ratio of the apparent absolute values in the depth direction is not changed, such that, when observing a stereoscopic image generated by radiographing a thin portion and a thick portion, the apparent ratio of the thickness between the stereoscopic images becomes equal to the actual ratio, making it possible to facilitate diagnosis at the time of medical diagnosis.

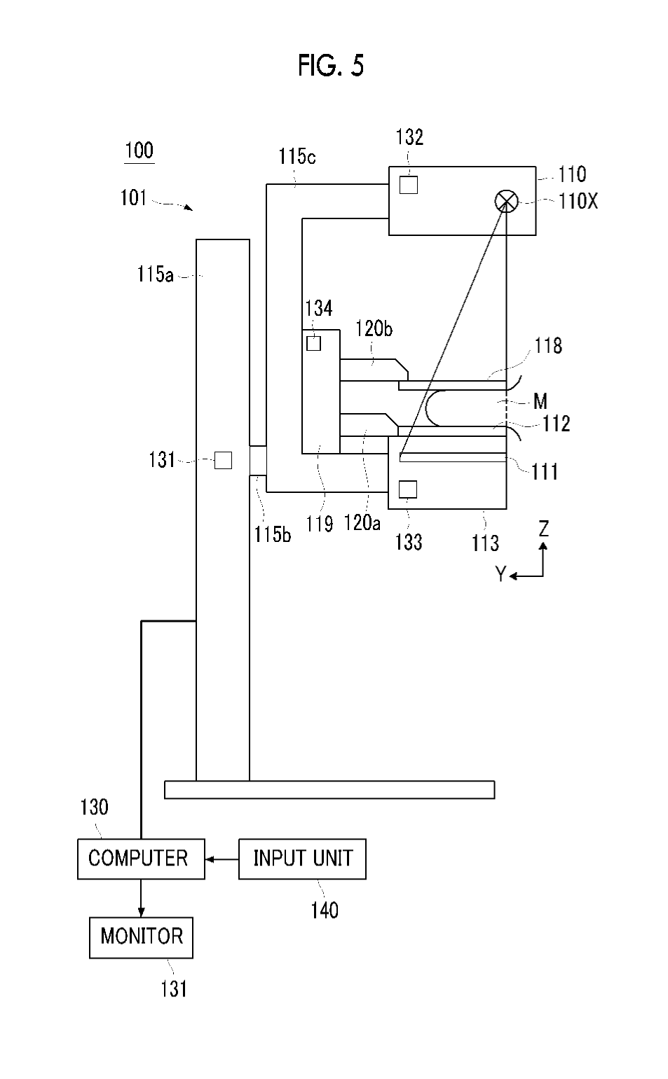

Although the radiation stereo image radiographing apparatus of the above-described embodiment can radiograph the entire subject P, the present invention can also be applied to a mammographic stereo image radiographing and display system. Next, a breast image radiographing apparatus using an embodiment of a breast image radiographing apparatus of the present invention will be described with reference to the drawings. FIG. 5 is a diagram showing the schematic configuration of the entire breast image radiographing apparatus of this embodiment.

As shown in FIG. 5, a breast image radiographing apparatus 100 of this embodiment includes a breast image radiographing device 101, a computer 130 which is connected to the breast image radiographing device 101, and a monitor 131 and an input unit 140 which are connected to the computer 130.

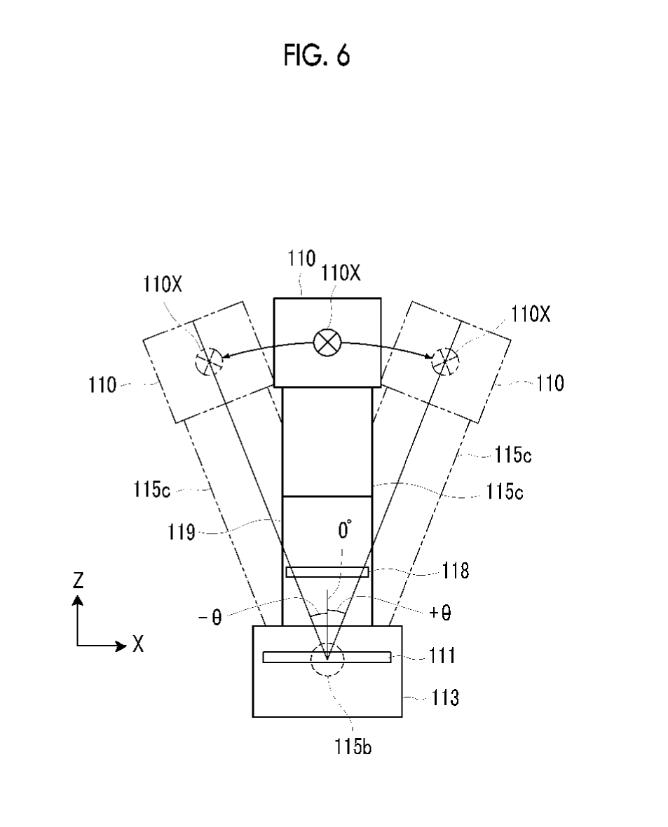

As shown in FIG. 5, the breast image radiographing device 101 includes a base 115a, a rotation shaft 115b which is movable in the up-down direction (Z direction) with respect to the base 115a and rotatable, and an arm portion 115c which is connected to the base 115a by the rotation shaft 115b. FIG. 6 shows the arm portion 115c when viewed from the right direction of FIG. 5.

The arm portion 115c has a C shape. A radiographing stand 113 is attached to one end of the arm portion 115c, and an irradiation unit 110 is attached to another end of the arm portion 115c to face the radiographing stand 113. The rotation and the movement in the up-down direction of the arm portion 115c are controlled by an arm controller 131 which is incorporated into the base 115a.

Inside the radiographing stand 113 are provided a radiation image detection unit 111, such as a flat panel detector, and a detector controller 133 which controls the reading of an electric charge signal from the radiation image detection unit 111. Inside the radiographing stand 113 is also provided a circuit board or the like on which a charge amplifier for converting an electric charge signal read from the radiation image detector 111a to a voltage signal, a correlated duplex sampling circuit for sampling a voltage signal output from the charge amplifier, or an AD conversion unit for converting a voltage signal to a digital signal, and the like are provided.

The radiographing stand 113 is configured to rotate with respect to the arm portion 115c. When the arm portion 115c has rotated with respect to the base 115a, the direction of the radiographing stand 113 can be fixed with respect to the base 115a.

The radiation image detector 111a can repeatedly record and read radiation images, and may be a so-called direct-type radiation image detector which directly receive radiation to generate electric charges or a so-called indirect-type radiation image detector which temporarily converts radiation to visible light and converts visible light to electric charge signals. As a method of reading radiation image signals, it is preferable to use a so-called TFT reading method which turns on/off a TFT (thin film transistor) switch to read radiation image signals or a so-called light reading method which irradiates read light to read radiation image signals. The present invention is not limited thereto, and other methods may be used.

In the irradiation unit 110, a radiation source 110X and a radiation source driving unit 132 are accommodated. The radiation source driving unit 132 controls the timing at which radiation is irradiated from the radiation source 110X and the radiation generation conditions (tube current, time, tube current-time product, and the like) in the radiation source 110X.

In the central portion of the arm portion 115c are provided a support 112 which is arranged above the radiographing stand 113 and on which a breast is placed, a support bearing portion 120a which supports the support, a compression plate 118 which compresses the breast from above, a compression plate bearing portion 120b which supports the compression plate 118, and a moving mechanism 19 which moves the support bearing portion 120a and the compression plate bearing portion 120b in the up-down direction (Z direction). The position of the support 112, the position of the compression plate 118, and the compressing pressure are controlled by the support/compression plate driving unit 134.

Although in this embodiment, as described above, both the support 112 and the compression plate 118 are movable in the up-down direction, the present invention is not limited thereto, and the position of the compression plate 118 may be fixed and only the support 112 may be movable in the up-down direction.

The input unit 140 and the monitor 131 have the same configuration as the input unit 40 and the monitor 31 of FIG. 2 in the radiation stereo image radiographing apparatus of the above-described embodiment, and description thereof will be omitted. The computer 130 substantially has the same configuration as the computer 30 in the radiation stereo image radiographing apparatus of the above-described embodiment, excluding the subject positioning unit 30c, and only different portions will be described.

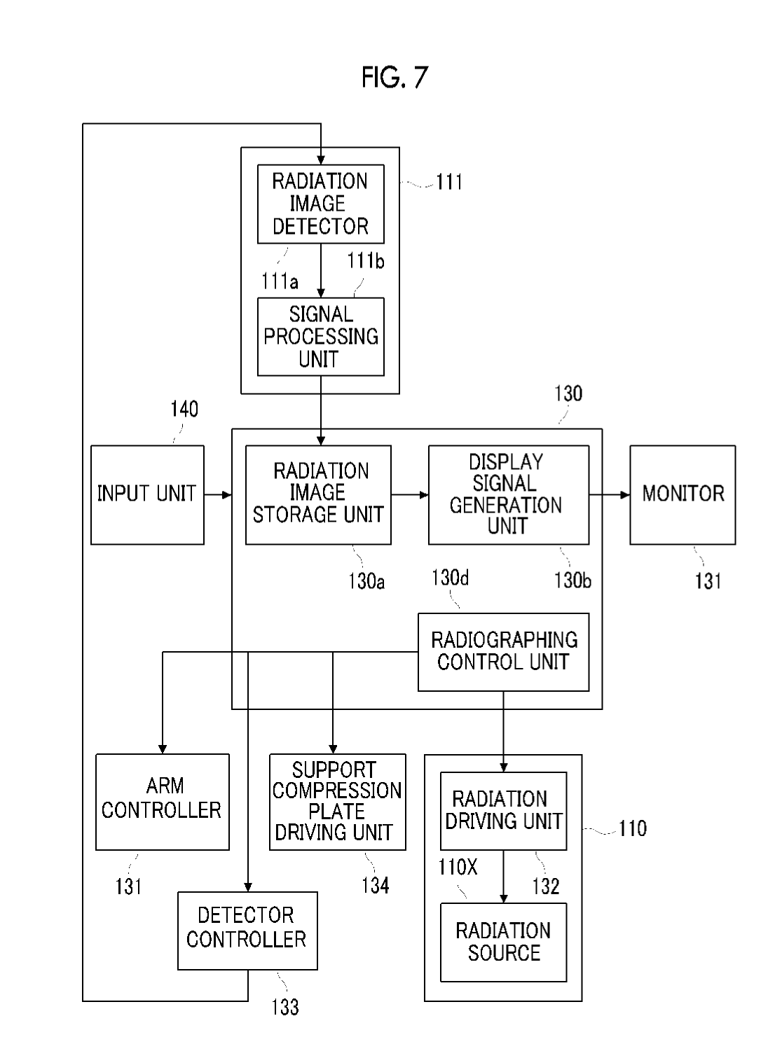

The computer 130 includes a central processing unit (CPU), a storage device, such as a semiconductor memory, a hard disk, and an SSD, and the like, which constitute a radiation image storage unit 130a, a display signal generation unit 130b, and a radiographing control unit 130d shown in FIG. 7.

The radiographing control unit 130d outputs a predetermined control signal to various controllers 131 and 133 and various driving units 132 and 134. A specific control method will be described below.

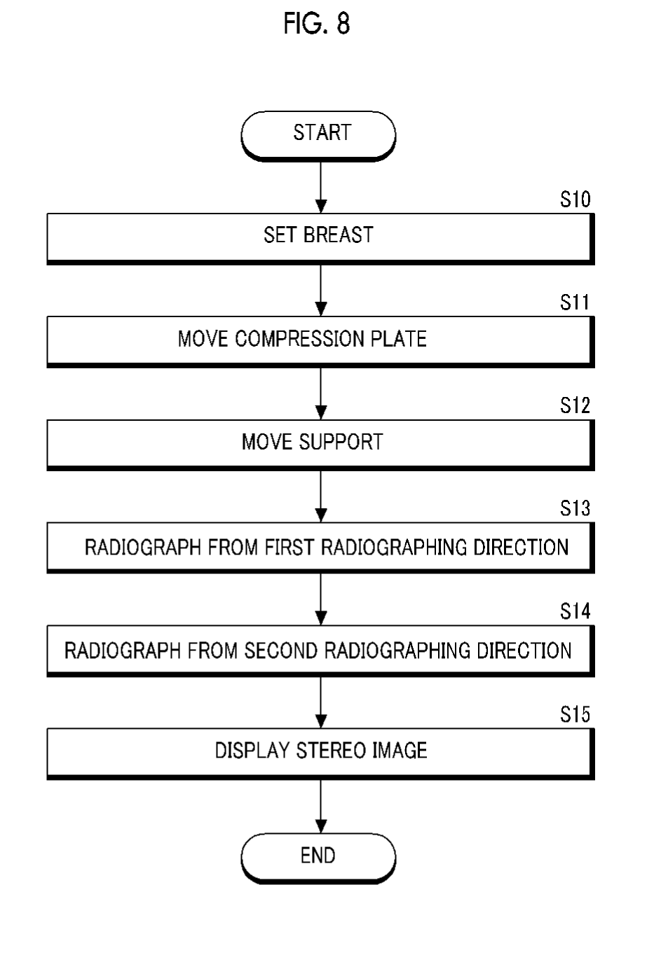

Next, the action of the breast image radiographing apparatus of this embodiment will be described with reference to a flowchart of FIG. 8.

First, the breast M of a patient is placed on the radiographing stand 113 (S10), and the compression plate 118 is moved by the moving mechanism 119 at a given distance defined in advance from the radiographing stand 113, that is, the radiation image detection unit 111 and fixed at that position (S11).

Next, the support 112 is moved in the up direction, that is, toward the compression plate 118 by the moving mechanism 119, such that the breast M is compressed against the compression plate 118 from the support 11 side and a predetermined pressure is imposed thereon (S12).

In this way, the breast M is not compressed from above by the movement of the compression plate 118 but compressed from below by the movement of the support 11, such that the position of the upper surface of the breast M is maintained at a given position. For this reason, when radiographing the breasts M of a plurality of human subjects P having different thicknesses t, the distance between the position of the compression plate 118 and the radiation image detection unit 111 becomes a given distance regardless of the breast M of which human subject P is radiographed, and even when the thickness t of the breast M differs, in the stereoscopic image of any breast M, the projection amount can be mad uniform. Simultaneously, since the convergence angle θ does not change or the shift amounts of the left-eye radiation image and the right-eye radiation image do not change at the time of the display of the stereoscopic image, there is no change in the parallax of the left-eye radiation image and the right-eye radiation image. For this reason, in the stereoscopic image of any breast M, the sense of depth in the stereoscopic image can be made uniform.

Next, in the input unit 140, after various radiographing conditions are input by the radiographer, a radiographing start instruction is input.

If there is a radiographing start instruction in the input unit 140, radiographing of a stereo image of the breast M is carried out (S13). Specifically, first, the radiographing control unit 130d reads the convergence angle θ set in advance for radiographing a stereo image and outputs information regarding the read convergence angle θ to the arm controller 131. In this embodiment, it is assumed that, for example, θ=±2° is stored in advance as information regarding the convergence angle θ at that time.

If the information regarding the convergence angle θ output from the radiographing control unit 130d is received by the arm controller 131, as shown in FIG. 6, the arm controller 131 outputs a control signal to rotate the arm portion 115c by +θ° with respect to the direction perpendicular to the radiographing stand 113 based on the information regarding the convergence angle θ. That is, in this embodiment, a control signal is output to rotate the arm portion 115c by +2° with respect to the direction perpendicular to the radiographing stand 113.

The arm portion 115c rotates by +2° in accordance with the control signal output from the arm controller 131. Subsequently, the radiographing control unit 130d outputs a control signal to the radiation source driving unit 132 and the detector controller 133 to irradiate radiation and to read radiation image signals. With the control signal, radiation is emitted from the radiation source 110X, a radiation image obtained by radiographing the breast from the +2° direction is detected by the radiation image detection unit 111, and a radiation image signal is read by the detector controller 133 and stored in the radiation image storage unit 130a of the computer 130 after having been subjected to predetermined signal processing.

Next, as shown in FIG. 6, the arm controller 131 temporarily returns the arm portion to the initial position and outputs a control signal to rotate the arm portion by −θ° with respect to the direction perpendicular to the radiographing stand 113 (S14). That is, in this embodiment, a control signal is output to rotate the arm portion 115c by −2° with respect to the direction perpendicular to the radiographing stand 113.

The arm portion 115c rotates by −2° in accordance with the control signal output from the arm controller 131. Subsequently, the radiographing control unit 130d outputs a control signal to the radiation source driving unit 132 and the detector controller 133 to irradiate radiation and to read radiation images. With the control signal, radiation is emitted from the radiation source 110X, a radiation image obtained by radiographing the breast from the −2° direction is detected by the radiation image detection unit 111, and a radiation image signal is read by the detector controller 133 and stored in the radiation image storage unit 130a of the computer 130 after having been subjected to predetermined signal processing.

Two radiation image signals for left-eye and right-eye stereo images are output to the display signal generation unit 130b. The display signal generation unit 130b generates a display control signal based on the input radiation image signals and outputs the display control signal to the monitor 131. The monitor 131 displays the radiation images based on the input display control signal, thereby displaying the left-eye stereo image and the right-eye stereo image (S15). The left-eye stereo image and the right-eye stereo image may be displayed simultaneously or switchingly.

When the observer observes the monitor 131, when the projection amount of the stereoscopic image is excessively projected or retracted and optimum stereoscopic view may not be performed, the shift amounts of the left-eye and right-eye stereo images may be shifted to adjust the projection amount. At this time, in this embodiment, with regard to a plurality of stereoscopic images obtained by radiographing the breasts M of a plurality of human subjects P having different thicknesses, the projection amount in each stereoscopic image is made uniform, such that the shift amount at the time of the adjustment has the same value between the stereoscopic images, thereby maintaining the projection amount of each stereoscopic image uniform. Simultaneously, a change in the parallax amount between the left-eye and right-eye stereo image for generating each stereoscopic image can be made uniform in each stereoscopic image.

Therefore, in the stereoscopic images of the breasts M of a plurality of human subjects P having different thicknesses, it is possible to adjust the projection amount while maintaining the projection amount and the sense of depth between the stereoscopic images uniform.

As described above, according to the breast image radiographing apparatus 100 and the breast image radiographing and displaying method of this embodiment, when radiographing the breasts M of a plurality of human subjects P having different thicknesses t, the distance between the position of the compression plate 118 and the radiation image detection unit 111 becomes a given distance regardless of the breast M of which human subject is radiographed, and even when the thickness of the breast M differs, in the stereoscopic image of any breast M, the projection amount can be made uniform. Simultaneously, since the convergence angle θ does not change or the shift amounts of the left-eye radiation image and the right-eye radiation image do not change at the time of the display of the stereoscopic image, there is no change in the parallax of the left-eye radiation image and the right-eye radiation image. For this reason, in the stereoscopic image of any breast M, the sense of depth in the stereoscopic image can be made uniform.

The sense of depth, that is, the ratio of the apparent absolute values in the depth direction does not change, such that, when observing the stereoscopic image of the breast M generated by radiographing a thin portion having a thickness t and a thick portion having a thickness t, the apparent ratio of the thickness between the stereoscopic images becomes equal to the actual ratio, making it possible to facilitate diagnosis at the time of medical diagnosis.

The present invention is not limited to the contents of the above-described embodiments, and may be appropriately changed without departing from the spirit and scope of the invention.

What is claimed is:

1. A radiation image radiographing apparatus comprising:
an irradiation unit which irradiates radiation onto a subject from different radiographing directions;
a radiation image detection unit which detects radiation having been irradiated by the irradiation unit and having passed through the subject;
a subject positioning unit which controls the position of the subject such that the distance between the end surface of the subject facing the irradiation unit and the radiation image detection unit becomes a given distance.

2. The apparatus according to claim 1, further comprising:
a subject position detector which detects the end surface of the subject facing the irradiation unit.

3. The apparatus according to claim 2,
wherein the subject position detection unit has an optical sensor, and
the end surface is detected by the optical sensor.

4. The apparatus according to claim 1, further comprising:
a support portion which supports the subject; and
a support portion driving unit which moves the support portion between the irradiation unit and the radiation image detection unit,
wherein the subject positioning unit controls the support portion driving unit such that the distance between the position of the end surface of the subject and the radiation image detection unit becomes the given distance.

5. The apparatus according to claim 2, further comprising:
a support portion which supports the subject;
a support portion driving unit which moves the support portion between the irradiation unit and the radiation image detection unit,
wherein the subject positioning unit controls the support portion driving unit such that the distance between the position of the end surface of the subject and the radiation image detection unit becomes the given distance.

6. The apparatus according to claim 3, further comprising:
a support portion which supports the subject; and
a support portion driving unit which moves the support portion between the irradiation unit and the radiation image detection unit,
wherein the subject positioning unit controls the support portion driving unit such that the distance between the position of the end surface of the subject and the radiation image detection unit becomes the given distance.

7. The apparatus according to claim 1, further comprising:
a display device which acquires the radiation images in the radiographing directions detected by the radiation image detection unit and displays a stereoscopic image using the acquired radiation images in the radiographing directions.

8. A breast image radiographing apparatus comprising:
an irradiation unit which irradiates radiation onto the breast of a human subject from different radiographing directions;
a radiation image detection unit which detects radiation having been irradiated by the irradiation unit and having passed through the breast;
a support on which the breast is placed;
a compression plate which is provided between the irradiation unit and the radiation image detection unit, and fixed at a given distance from the radiation image detection unit to compress the breast on the support; and
a support driving unit which moves the support toward the compression plate to compress the breast.

9. The apparatus according to claim 8, further comprising:
a display device which acquires radiation images in the radiographing directions detected by the radiation image detection unit and displays a stereoscopic image using the acquired radiation images in the radiographing directions.

10. A radiation image radiographing and displaying method which irradiates radiation onto a subject from different radiographing directions by an irradiation unit to acquire radiation images in the radiographing directions detected by the radiation image detection unit, and displays a stereoscopic image using the acquired radiation images in the radiographing directions,
wherein the position of the subject is controlled such that the distance between the position of the end surface of the subject facing the irradiation unit and the radiation image detection unit becomes a given distance,
in a state where the position of the subject is controlled, the radiation images in the radiographing directions are acquired, and
the stereoscopic image is displayed using the acquired radiation images.

11. The method according to claim 10,
wherein the position of the end surface of the subject facing the irradiation unit is detected.

12. The method according to 11,
wherein the position of the end surface of the subject is detected by an optical sensor.

13. The method according to claim 10,
wherein the subject is supported by a support portion, and
the support portion is moved between the irradiation unit and the radiation image detection unit to control the position of the subject such that the distance between the position of the end surface of the subject and the radiation image detection unit becomes the given distance.

14. The method according to claim 11,
wherein the subject is supported by a support portion, and
the support portion is moved between the irradiation unit and the radiation image detection unit to control the position of the subject such that the distance between the position of the end surface of the subject and the radiation image detection unit becomes the given distance.

15. The method according to claim 12,
wherein the subject is supported by a support portion, and
the support portion is moved between the irradiation unit and the radiation image detection unit to control the position of the subject such that the distance between the position of the end surface of the subject and the radiation image detection unit becomes the given distance.

16. A breast image radiographing and displaying method which irradiates radiation onto the breast of a human subject from different radiographing directions by an irradiation unit to acquire radiation images in the radiographing direction detected by a radiation image detection unit, and displays a stereoscopic image using the acquired radiation images in the radiographing directions,
wherein a support is provided on which the breast is placed,
a compression plate is provided between the irradiation unit and the radiation image detection unit, and fixed at a given distance from the radiation image detection unit to compress the breast on the support,
the support is moved toward the compression plate to compress the breast,
in a state where the breast is compressed, the radiation images in the radiographing directions are acquired, and
the stereoscopic image is displayed using the acquired radiation images.
